(19) United States
(12) Patent Application Publication
(10) Pub. No.: US 2006/0068009 A1
(43) Pub. Date: Mar. 30, 2006

(54) Modified release ibuprofen dosage form

(75) Inventors: Michael Hite, Seattle, WA (US); Cathy Federici, Seattle, WA (US); Alan Brunelle, Woodinville, WA (US); Stephen Turner, Snoqualmie, WA (US)

Correspondence Address: RATNERPRESTIA, P O BOX 980, VALLEY FORGE, PA, 19482-0980, US

(73) Assignee: SCOLR Pharma, Inc.

(21) Appl. No.: 11/238,802

(22) Filed: Sep. 29, 2005

Related U.S. Application Data
(60) Provisional application No. 60/614,932, filed on Sep. 30, 2004.
(60) Provisional application No. 60/689,631, filed on Jun. 10, 2005.

(51) Int. Cl. A61K 9/22

(52) U.S. Cl. 424/468

(57) ABSTRACT

The present invention is a solid dosage form for oral administration of ibuprofen comprising a modified release formulation of ibuprofen which provides an immediate burst effect and thereafter a sustained release of sufficient ibuprofen to maintain blood levels at least 6.4 μg/ml over an extended period of at least 8 hours following administration of a single dose. The dosage form releases ibuprofen at a rate sufficient to initially deliver a effective amount of ibuprofen within about 2.0 hours following administration. The dosage form then subsequently delivers the remaining amount of ibuprofen at a relatively constant rate sufficient to maintain a level of ibuprofen over a predetermined delivery period of for at least 8 hours.

### CROSS REFERENCE TO RELATED APPLICATIONS

The present invention claims the benefit of U.S. Provisional Applications Nos. 60/614,932, filed Sep. 30, 2004 and 60/689,631, filed Jun. 10, 2005.

### BACKGROUND OF THE INVENTION

Ibuprofen is 2-(4-isobutylphenyl)propionic acid and is a non-steroidal anti-inflammatory compound (NSAID), which exhibits high levels of anti-inflammatory, analgesic and antipyretic activities necessary for the effective treatment of rheumatoid arthritis and osteo-arthritis and other inflammatory conditions. Most dosage forms of ibuprofen are immediate release dosage forms that provide rapid onset of therapeutic action, then rapidly declining levels of active ingredient, necessitating repeated dosing. They do not maintain therapeutic levels from one treatment over an extended period of time. Repeat dosing is thus required at intervals of four to six hours. Formulations that claim extended release fail to have an initial burst of the drug and thus exhibit substantial delay between administration and the achievement of an effective therapeutic blood level. Therefore, a need exists for a solid dosage form, for example a compressed tablet, which provides an initial burst of released ibuprofen, leading to prompt onset of action, then thereafter provides a sustained release of sufficient ibuprofen to maintain beneficial blood levels of ibuprofen over an extended period of 8 or more hours.

### SUMMARY OF THE INVENTION

In accordance with the foregoing, we have provided a solid dosage form for oral administration of ibuprofen comprising a modified release formulation of ibuprofen which provides an immediate burst effect and thereafter a sustained release of sufficient ibuprofen to maintain blood levels at least 6.4 μg/ml over an extended period of at least 8 hours following administration of a single dose.

More particularly, the invention comprises a solid dosage form for oral administration comprising a hydrophilic polymer, a pharmaceutically effective amount of ibuprofen in the range of 300 mg to 800 mg uniformly dispersed in the polymer, a dissolution additive dispersed in the polymer in an amount in the range of 10% to 35% by weight of the ibuprofen, and a formulation additive dispersed in the polymer in an amount of 15% to 75% by weight of the ibuprofen. The dosage form releases ibuprofen at a rate sufficient to initially deliver a effective amount of ibuprofen within about 2.0 hours following administration. The dosage form then subsequently delivers the remaining amount of ibuprofen at a relatively constant rate sufficient to maintain a level of ibuprofen over a predetermined delivery period of for at least 8 hours.

As used herein, a relative constant rate refers to a substantially linear relationship shown in the examples following the initial burst (up to about 2 hours) between percentage released and elapsed time.

### BRIEF DESCRIPTION OF THE DRAWINGS

FIG. 1: In-vitro dissolution of Example 1

FIG. 2: In-vitro dissolution of Example 2

FIG. 3: In-vitro dissolution of Example 3

FIG. 4: In-vitro dissolution of Example 4

FIG. 5: In-vitro dissolution of Example 5

FIG. 6: In-vitro dissolution of Example 6

FIG. 7: In-vitro dissolution of Example 7

FIG. 8: In-vitro dissolution of Example 8

FIG. 9: In-vitro dissolution of Example 9

FIG. 10: In-vitro dissolution of Example 10

FIG. 11: In-vitro dissolution of Example 11

FIG. 12: In-vitro dissolution of Example 12

FIG. 13: In-vitro dissolution of Example 13

FIG. 14: In-vitro dissolution of Example 14

FIG. 15: In-vitro dissolution of Example 15

FIG. 16: In-vitro dissolution of Example 16

FIG. 17: In-vitro dissolution of Examples 17 and 18

FIG. 18: In-vitro dissolution of BRUFEN RETARD, an extended release form of Ibuprofen available for sale in Europe.

FIG. 19: In-vivo data from comparison of present invention versus Motrin®

### DETAILED DESCRIPTION OF THE INVENTION

The present invention is further illustrated and described by reference to the following disclosure, examples and discussion below. In the examples and discussion which follow, the use of particular polymers, electrolytes, additives, fillers and tableting aids are provided by way of example only and are not intended to limit the scope of this invention. Although the invention is illustrated and described herein with reference to specific embodiments, the invention is not intended to be limited to the details shown. Rather, various modifications may be made in the details within the scope and range of equivalents of the claims and without departing from the invention.

The ibuprofen content of the dosage form may be between in the range about 300 mg and about 800 mg per dosage unit, preferably about 300, 400 or 600 mg per unit dosage form. Also contemplated is using prodrugs of ibuprofen such as ibuprofen-lysine and ibuprofen-arginine. If a smaller dosage form is desired, a single dose of ibuprofen may be divided between multiple, for example two to three, dosage units, such as tablets, which may be administered at substantially the same time. The dosage form may comprise from about 25% to about 75% by weight ibuprofen.

The hydrophilic polymer used in the dosage form may be selected from a wide variety of hydrophilic polymers. Hydrophilic polymers suitable for use in the sustained release formulation include: one or more natural or partially or totally synthetic hydrophilic gums such as acacia, gum tragacanth, locust bean gum, guar gum, or karaya gum; modified cellulosic substances such as methylcellulose, hydroxy methylcellulose, hydroxypropyl methylcellulose, hydroxypropyl cellulose, hydroxyethylcellulose, or carboxyethylcellulose; proteinaceous substances such as agar, pectin, carrageenan, and alginates; and other hydrophilic polymers such as carboxypolymethylene, gelatin, casein, zein, bentonite, magnesium aluminum silicate, polysaccharides, modified starch derivatives, and other hydrophilic polymers known to those of skill in the art, or a combination of such polymers.

These hydrophilic polymers gel and dissolve slowly in aqueous acidic media thereby allowing the ibuprofen to diffuse from the gel in the stomach and gastrointestinal tract. Hydroxypropyl methylcellulose (HPMC) and other hydrophilic polymers mentioned above may be available in forms that have varying viscosity ratings. In general these polymers, or the combination of them, may be present in the dosage form alone or in combination in an amount or at a concentration in the range of 10% to 70% by weight of the ibuprofen present in the formulation, for example 15% to 50% or 15% to 33%, depending on the release pattern which is sought to be achieved with the particular dosage form.

One hydrophilic polymer useful in the present invention is HPMC K4M. This is a nonionic swellable hydrophilic polymer manufactured by “The Dow Chemical Company” under the tradename “Methocel.” HPMC K4M is also referred to as HPMC K4MP, in which the “P” refers to premium cellulose ether designed for controlled release formulations. The “4” in the abbreviation suggests that the polymer has a nominal viscosity (2% in water) of 4000. The percent of methoxyl and hydroxypropyl groups are 19-24 and 7-12, respectively. In its physical form, HPMC K4M is a free-flowing, off-white powder with a particle size limitation of 90%<100 mesh screen. A more complete list of HPMC is K100LVP, K15MP, K100MP, E4MP and E10MP CR with nominal viscosities of 100, 15000, 100000, 4000, and 10000 respectively.

The solid dosage form also includes at least one formulation additive such as one or more of a filler, a diluent or a compression aid. These are additives which aid in preparation or manufacture of the dosage form and for a tableted solid dosage form a tableting aid such as microcrystalline cellulose (MCC), such MCC 105 (particle size of about 20 μm), MCC 200 (particle size of about 180 μm) and MCC 302 (particle size of about 90 μm), silicified microcrystalline cellulose (MCC bonded to SiO₂), such as Prosolv90 (particle size of about 90 μm) and Prosolv50 (paricle size of about 50 μm), lactose, such as spray dried lactose (Lactopress®), dicalcium phosphate, silica or pregelatinized starch and combinations thereof may be incorporated into the formulation in an amount or at a concentration in the range of about 15% to about 75% by weight of the ibuprofen present in the dosage form. It is contemplated that various particle sizes of microcrystalline cellulose may be used if desired, for example two different particle sizes in which each of them are present in individual amounts in the range of 17% to 33% by weight of the ibuprofen present in the formulation. In one embodiment, one can pre-blend silica with ibuprofen or pre-blend silica and/or formulation additive MCC with ibuprofen.

In addition to formulation additives, the dosage form also contains at least one dissolution additive. Such additives which generally comprise a pore-forming, wetting or disintegration agent which facilitates dissolution of the dosage form. Such dissolution additives may be present in the dosage form at an amount or concentration in the range of about 10% to about 35% by weight of the ibuprofen, for example, at 10% to about 15%. The additive may suitably be selected from alkali metal salts, such as sodium and potassium carbonate; sodium carbonate, monohydrate; sodium bicarbonate; amino acids with neutral-to-basic side chains, such as glycine, alanine, valine, leucine, iso-leucine, cysteine, methionine, phenylalanine, proline, lysine, arginine, histidine, serine, threonine, asparagine, tryptophan, tyrosine and glutamine; conventional pharmaceutical disintegrants and combinations or mixtures thereof. Examples of such additives are sodium carbonate, glycine, arginine and croscarmellose sodium.

In addition to ibuprofen, multiple active ingredients are contemplated and may be present in the present dosage form. Combinations of ibuprofen with actives such as caffeine, psuedophedrine, aspirin, phenylephrine and/or sympathomemetics, analgesics, such as hydrocodone, and antihistamines are within the scope of the invention.

Favorable in vitro characteristics that lead to an acceptable in vivo efficacy are contemplated as 20% or greater release within 2.0 hour after oral administration or contact with an aqueous environment, followed by more gradual release over several hours, leading to release of at least 70% release in 8 to 12 hours following administration or contact with an aqueous environment. The method of determining in vitro release is using an agitated aqueous medium, such as stirring at 50 rpm in pH 7.2 KH₂PO₄ media; or surrogate methods using alternate pH media, such as 0.1N HCl or SGF @ pH 1.2 for an initial (30 min-2 hr period or using alternate hydrodynamic conditions such as 100 to 150 rpm for a period of 1-2 hrs).

The accepted range for minimal efficacy in vivo is from about 6.4 μg/ml to about 10 μg/ml mean ibuprofen blood concentration.

### EXAMPLES

The formulations of the invention are illustrated by the following examples. The use of particular polymers, electrolytes, additives, fillers and compression aids are not intended to limit the scope of this invention but are exemplary only.

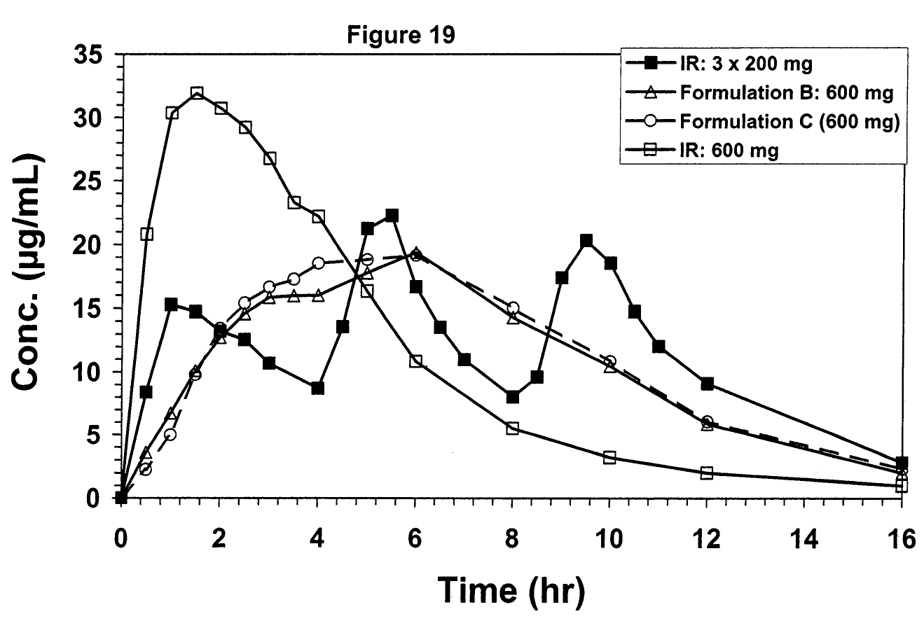

The solid dosage comprising a modified release formulation of the present invention was prepared and tested for both in vitro release and in vivo blood levels as described in Examples 1-20 below. In the in vivo testing, the dissolution rates of the subject dosage forms were compared against two commercially available tablets, one being an immediate release formulation of 200 mg of ibuprofen and the other being an immediate release 600 mg ibuprofen formulation. The solid dosage forms comprising the modified release formulation of the present invention demonstrated an initial burst similar to an immediate release tablet and a slower, more controlled release of ibuprofen over a eight hour period, as best seen in FIG. 19.

Unless otherwise noted, all in vitro release performance was evaluated in a type II dissolution apparatus in 900 mL KH₂PO₄ buffer, pH 7.2, at 50 rpm paddle speed.

### Example 1

In one embodiment, the formulation comprised ibuprofen, hydroxypropyl methylcellulose (HPMC K15M and HPMC K100LV), glycine and sodium carbonate, in which HPMC K15M was present at a concentration of 18% by weight of ibuprofen, HPMC K100LV was present at a concentration of 17% by weight of ibuprofen, glycine was present at a concentration of 2.5% by weight of ibuprofen, and sodium carbonate was present at a concentration of 17% by weight of ibuprofen within a monolithic compressed tablet. The specific formulations are as follows:

| Ex. 1a | mg | Ex. 1b | mg |
|---|---|---|---|
| Ibuprofen 90 grade | 600 | Ibuprofen 90 grade | 600 |
| HPMC K15M | 110 | HPMC K15M | 125 |
| HPMC K100LV | 100 | HPMC K100LV | 100 |
| MCC PH102 | 100 | MCC PH102 | 100 |
| Na₂CO₃, anhydrous | 150 | Na₂CO₃, anhydrous | 150 |
| Glycine | 15 | Glycine | 15 |
| Silica, Syloid 244 | 20 | Silica, Syloid 244 | 20 |
| Mg Stearate | 10 | Mg Stearate | 10 |
| Total: | 1105 | Total: | 1120 |

All ingredients were passed through a 30-mesh screen and blended with the remaining formulation components in a V-blender. The resulting powder was compressed into tablets using conventional compression techniques.

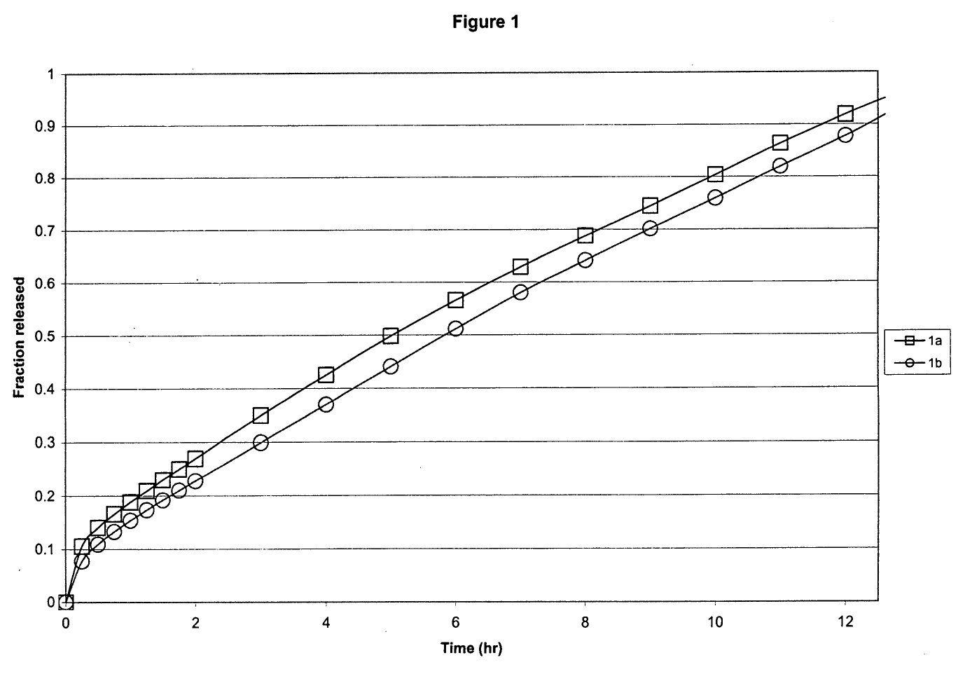

As shown in FIG. 1, the results of this Example demonstrate that the invention is capable of an in vitro release profile comprising a burst effect, followed by the sustained release of the remaining material, leading to in excess of 90% release in approximately 12 hours. This formulation thus overcomes one of the principle problems with many ibuprofen formulations which exhibit substantially less than complete release over an extended period of time.

### Example 2

In another embodiment, the formulation comprised ibuprofen, hydroxypropyl methylcellulose (HPMC K100M and HPMC K100LV), sodium carbonate, flow agents and tableting aids, in which HPMC K100M was present at a concentration of 17% by weight of ibuprofen, HPMC K100LV was present at a concentration of 17% by weight of ibuprofen and sodium carbonate was present at a concentration of 25% by weight of ibuprofen within a compressed monolithic tablet. The specific formula is as follows:

| Ex. 2 | mg |
|---|---|
| Ibuprofen | 600 |
| HPMC K100M | 100 |
| HPMC K100LV | 100 |
| Na₂CO₃, anhydrous | 150 |
| MCC PH102 | 150 |
| Silica, Syloid 244 | 20 |
| Mg Stearate | 10 |
| Total: | 1130 |

The formulation components were mixed in a V-blender. The resulting powder was compressed into tablets using conventional technologies. In this Example a combination of a medium to high viscosity HPMC and a low viscosity HPMC was used.

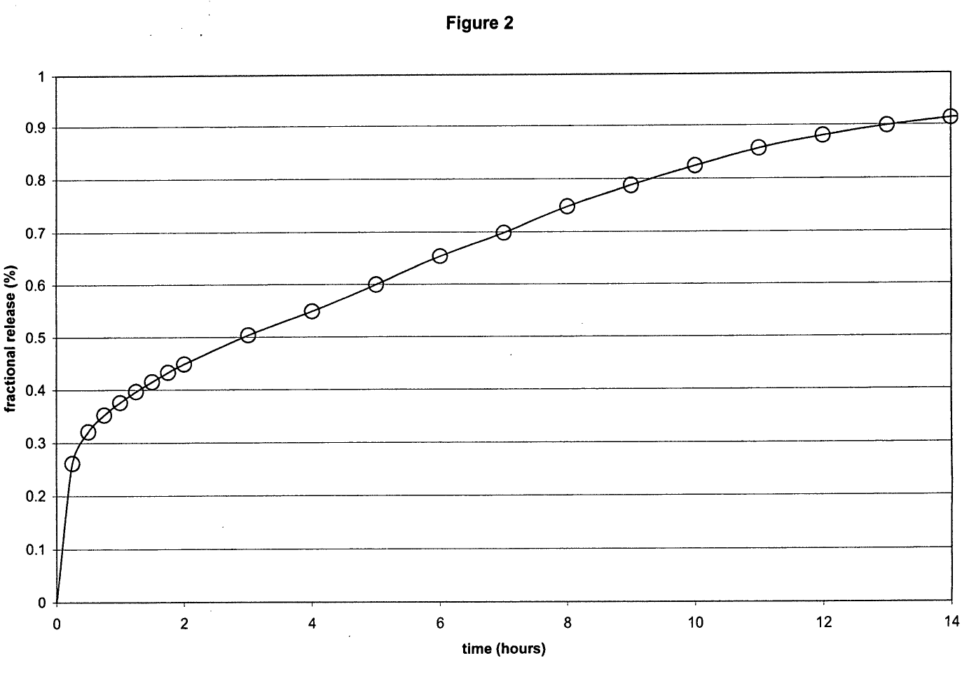

As shown in FIG. 2, the results of this Example demonstrate an in vitro release profile comprising a burst effect, followed by the sustained release of the remaining material. The burst effect provides release of 20% of ibuprofen within 2 hours, and the release of approximately 90% of the available ibuprofen over a period of 12 to 14 hours.

### Example 3

In another embodiment, the formulation comprised ibuprofen, hydroxypropyl methylcellulose (HPMC K15M and HPMC K100LV), sodium carbonate, flow agents and tableting aids, in which HPMC K100M was present at a concentration of 17% by weight of ibuprofen, HPMC K100LV was present at a concentration of 17% by weight of ibuprofen and sodium carbonate was present at a concentration of 25% by weight of ibuprofen within a compressed monolithic tablet.

| Ex. 3 | mg |
|---|---|
| Ibuprofen | 600 |
| HPMC K15M | 100 |
| HPMC K100LV | 100 |
| MCC PH102 | 100 |
| Na₂CO₃, anhydrous | 150 |
| Glycine | 15 |
| Silica, Syloid 244 | 20 |
| Mg Stearate | 10 |
| Total: | 1095 |

The formulation components were mixed in a V-blender. The resulting powder was compressed into tablets using conventional compression technology. In this Example a combination of a medium to high viscosity HPMC and a low viscosity HPMC was used.

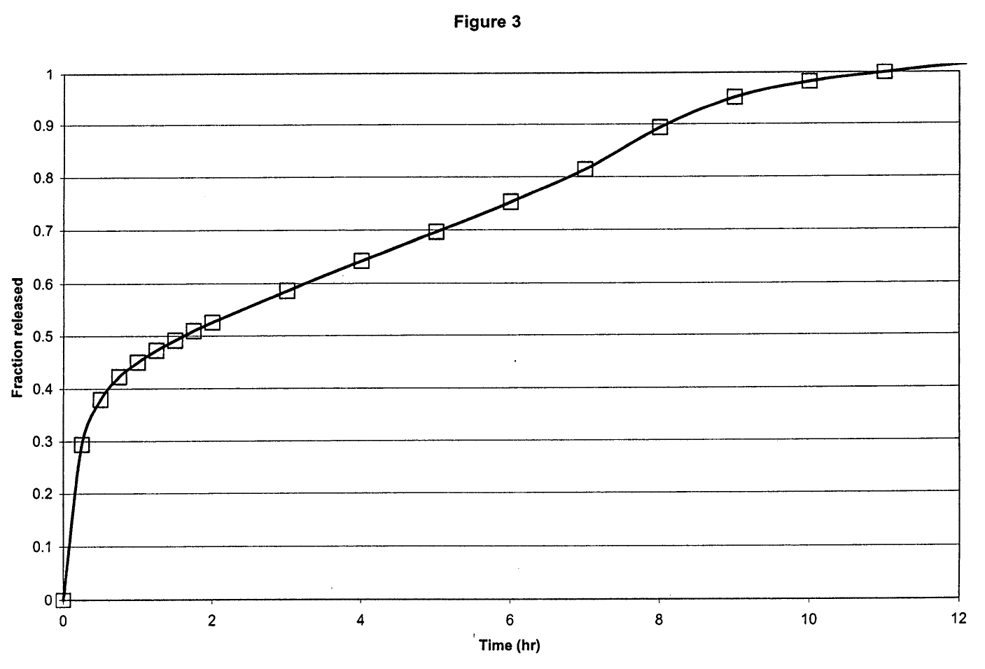

As shown in FIG. 3, the results of this Example demonstrate an in vitro release profile comprising a burst effect providing release of 20% of ibuprofen within 2 hours, followed by the sustained release of the remaining material evidencing release of 100% of the ibuprofen present in about 11 hours and greater than 90% in approximately 8 hours.

### Example 4

In another embodiment, the formulation comprised ibuprofen, hydroxypropyl methylcellulose (HPMC K100M and HPMC K100LV), sodium carbonate, flow agents and tableting aids, in which HPMC K100M was present at a concentration of 17% by weight of ibuprofen, HPMC K100LV was present at a concentration of 17% by weight of ibuprofen, and sodium carbonate was present at a concentration of 25% by weight of ibuprofen within a compressed monolithic tablet.

| Ex. 4 | mg |
|---|---|
| Ibuprofen | 600 |
| HPMC K100M | 100 |
| HPMC K100LV | 100 |
| MCC PH102 | 100 |
| Na₂CO₃, anhydrous | 150 |
| Silica, Syloid 244 | 20 |
| Mg Stearate | 10 |
| Total: | 1080 |

The formulation components were mixed in a V-blender. The resulting powder was compressed into tablets using conventional technologies. In this Example a combination of a medium to high viscosity HPMC and a low viscosity HPMC was used.

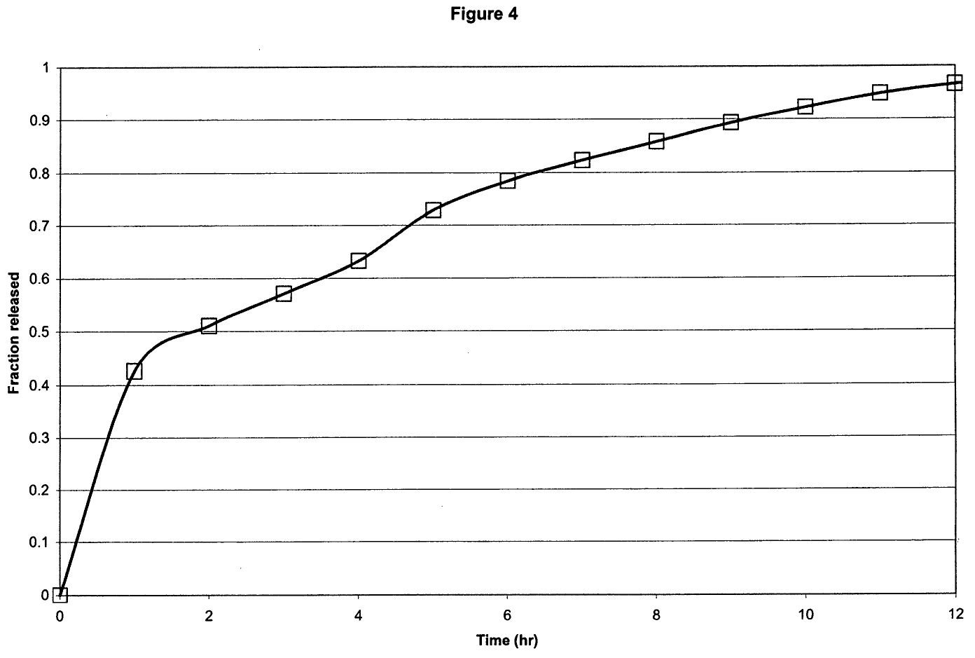

As shown in FIG. 4, the results of this Example demonstrate an in vitro release profile comprising a burst effect, followed by the sustained release of the remaining material. 20% of ibuprofen was released within 2 hours, followed by gradual sustained release, resulting in approximately 95% release after 12 hours.

### Example 5

In another embodiment, the formulation comprised ibuprofen, hydroxypropyl methylcellulose (HPMC K100M), polyethylene oxide (PEO WSRN 301), sodium carbonate, glycine, flow agents and tableting aids, in which HPMC was present at a concentration of 33% by weight of ibuprofen, glycine was present at a concentration of 8.25% by weight of ibuprofen and sodium carbonate was present at a concentration of 25% by weight of ibuprofen within a compressed monolithic tablet.

| Ex. 5 | mg |
|---|---|
| Ibuprofen | 600 |
| PEO 301 | 50 |
| HPMC K100M | 100 |
| MCC PH102 | 100 |
| Na₂CO₃, anhydrous | 150 |
| Glycine | 20 |
| Silica, Syloid 244 | 20 |
| Mg Stearate | 10 |
| Total: | 1050 |

The formulation components were mixed in a V-blender. The resulting powder was compressed into tablets using conventional compression technology.

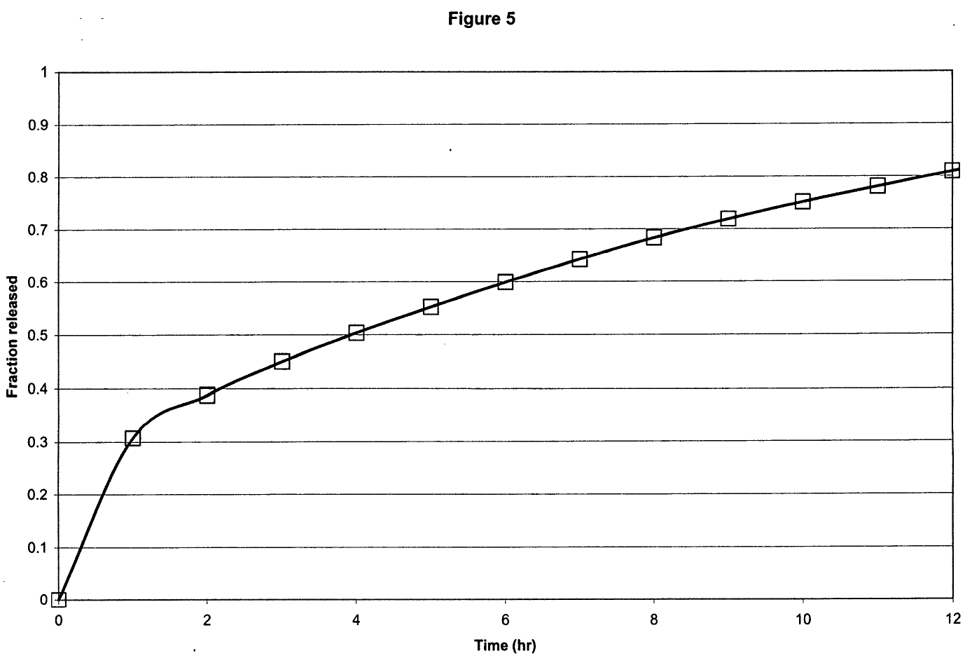

As shown in FIG. 5, the results of this Example demonstrate an in vitro release profile comprising a burst effect, followed by the sustained release of the remaining material. For this formulation 20% of ibuprofen was released within 2 hours, but incomplete release was evidenced after 12 hours.

### Example 6

In another embodiment, the formulation comprised ibuprofen, hydroxypropyl methylcellulose (HPMC K15M), potassium carbonate, flow agents and tableting aids, in which HPMC was present at a concentration of 33% by weight of ibuprofen, and potassium carbonate was present at a concentration of 17% by weight of ibuprofen within a compressed monolithic tablet.

| Ex. 6 | mg |
|---|---|
| Ibuprofen 90 grade | 600 |
| MCC PH 105 | 210 |
| HPMC K15M Prem | 190 |
| MCC PH 200 | 100 |
| K₂CO₃ anhydrous | 100 |
| | 1200 |

The formulation components were mixed in a V-blender. The resulting powder was compressed into tablets using conventional compression technology.

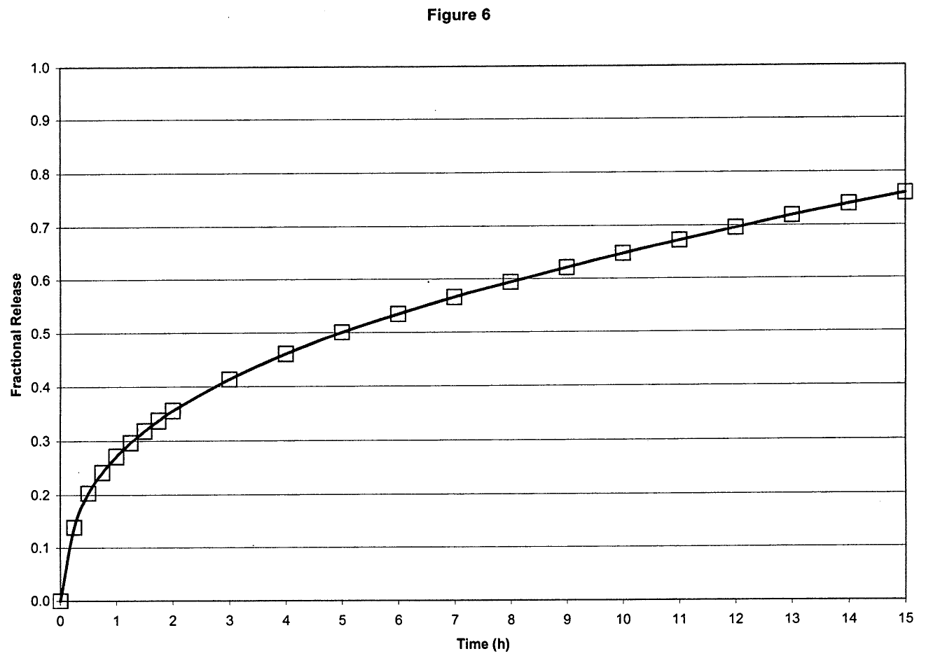

As shown in FIG. 6, the results of this Example demonstrate an in vitro release profile comprising a burst effect, followed by the sustained release of the remaining material. 20% of ibuprofen was released in under 2 hours, and release was thereafter sustained over a period of 15 hours. However, incomplete release was exhibited by the dosage form.

### Example 7

In this embodiment, the formulation comprised ibuprofen, hydroxypropyl methylcellulose (HPMC K15M), sodium carbonate, microcrystalline cellulose (MCC PH105 and MCC PH200), in which HPMC was present at a concentration of 33% by weight of ibuprofen, sodium carbonate was present at a concentration of 17% by weight of ibuprofen, MCC PH105 was present at a concentration of 33%, and MCC PH200 was present at a concentration of 17% within a compressed monolithic tablet.

| Ex. 7 | Mg |
|---|---|
| Ibuprofen 90 grade | 600 |
| HPMC K15M Prem | 190 |
| MCC PH 105 | 210 |
| MCC PH 200 | 100 |
| Na₂CO₃ anhydrous | 100 |
| | 1200 |

All ingredients were passed through a 30-mesh screen. The ibuprofen and the MCC 105 were blended in a V-blender. The resulting homogenous pre-blend was granulated with water, dried and subsequently blended with the remaining formulation components in a V-blender. The resulting powder was compressed into tablets using conventional compression technology.

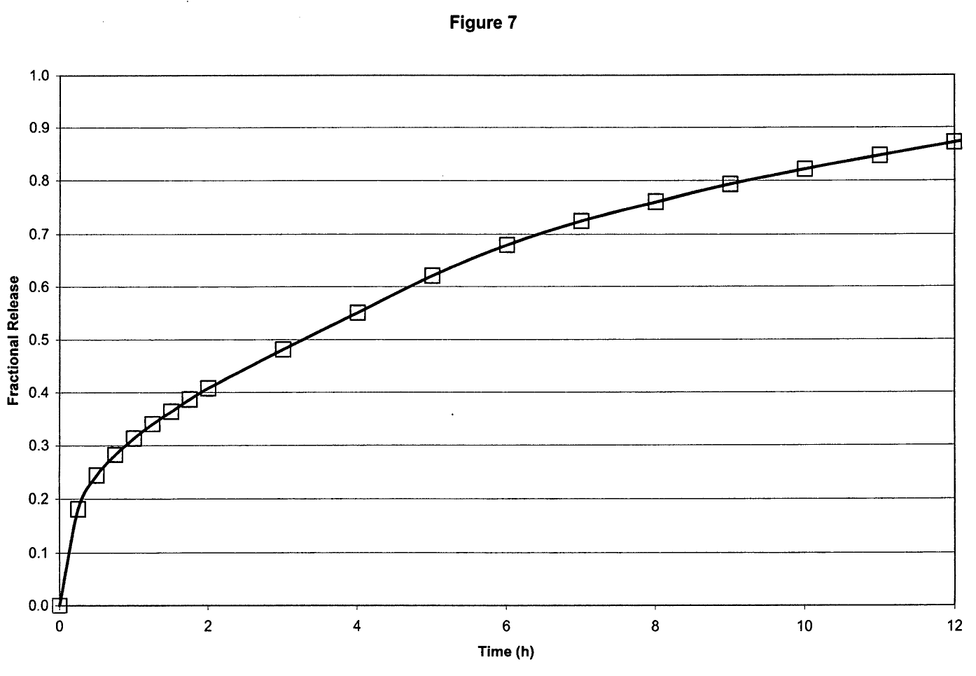

As shown in FIG. 7, this Example demonstrates an in vitro release profile comprising a burst effect, followed by the sustained release of the remaining material. The burst effect releases 20% of ibuprofen in under 2 hour, followed by relatively constant release over the next 10-12 hours and resulting in approximately 90% release after 12 hours.

### Example 8

In the embodiment of Example 1, the tablet resulting from the formulation was split into two equal parts, and both sections were placed into a dissolution vessel.

| Ex. 8 | mg |
|---|---|
| Ibuprofen 90 grade | 600 |
| HPMC K15M | 110 |
| HPMC K100LV | 100 |
| MCC PH102 | 100 |
| Na₂CO₃, anhydrous | 150 |
| Glycine | 15 |
| Silica, Syloid 244 | 20 |
| Mg Stearate | 10 |
| Total: | 1105 |

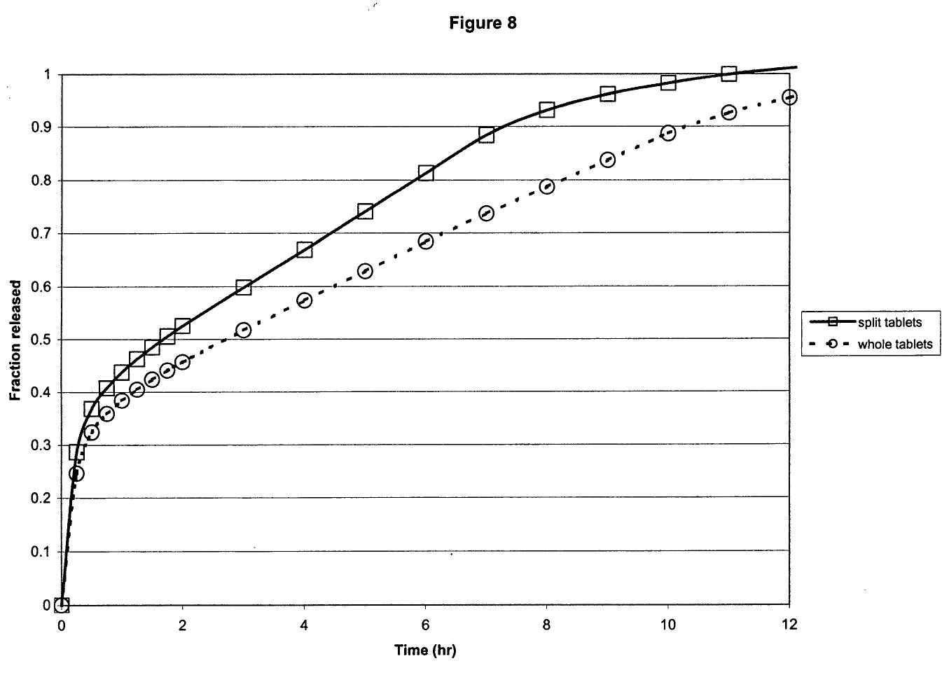

As shown in FIG. 8, the results of this Example demonstrates an in vitro release profile comprising a burst effect, followed by the sustained release of the remaining material, even when split into sections after tableting. In each case 20% of ibuprofen was released in less than one hour and substantially all the ibuprofen had been released at about 12 hours.

### Example 9

In one embodiment, the formulation comprised ibuprofen, hydroxypropyl methylcellulose (HPMC K15M), sodium carbonate, microcrystalline cellulose (MCC PH 302), in which HPMC was present at a concentration of 33% by weight of ibuprofen, sodium carbonate was present at a concentration of 18% by weight of ibuprofen, and MCC PH 302 was present at a concentration of 33% within a compressed monolithic tablet.

| Ex. 9 | mg |
|---|---|
| Ibuprofen 90 grade | 300 |
| HPMC K15M Prem | 100 |
| MCC PH 302 | 100 |
| Na₂CO₃ anhydrous | 50 |
| Glycine | 7.5 |
| Silica | 5.5 |
| Total: | 563 |

All ingredients were passed through a 30-mesh screen and blended in a V-blender. The resulting homogenous pre-blend was granulated with water, dried and subsequently blended with the remaining formulation components in a V-blender. The resulting powder was compressed into tablets using conventional technologies.

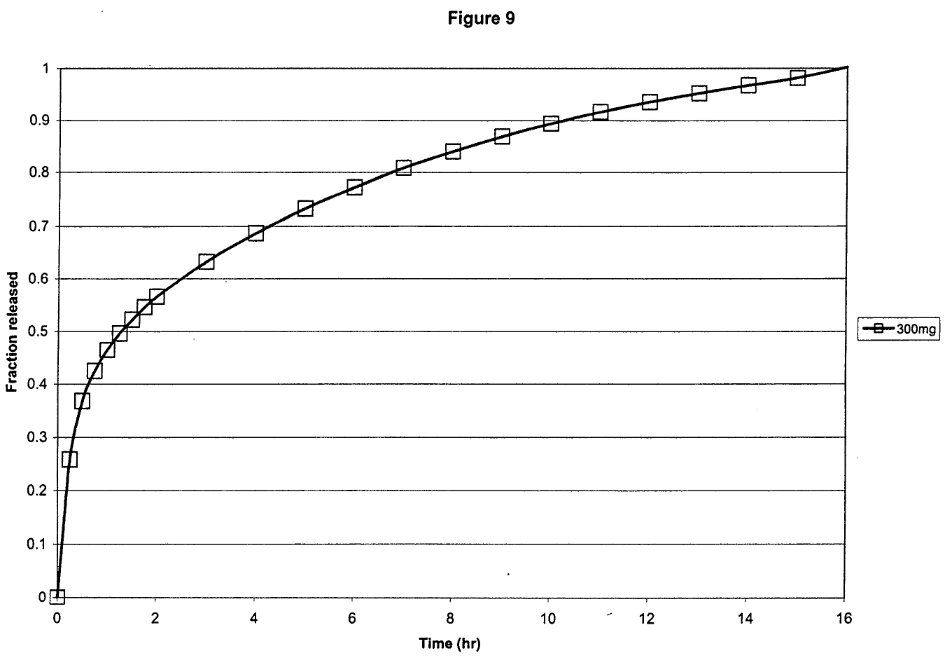

As shown in FIG. 9, the results of this Example demonstrate an in vitro release profile comprising a burst effect, followed by the sustained release of the remaining material. 20% of ibuprofen was released within 2 hours, about 90% release was obtained in about 9 hours followed by 100% release in under 16 hours.

### Example 10

In another embodiment, the formulation comprised ibuprofen, hydroxypropyl methylcellulose (HPMC K4M), flow agents and tableting aids, in which HPMC K4M was present at a concentration of 32% by weight of ibuprofen, and arginine was present at a concentration of 17% by weight of ibuprofen within a compressed monolithic tablet.

| Ex. 10 | mg |
|---|---|
| Ibuprofen 90 grade | 600 |
| Silica | 5.5 |
| MCC PH 105 | 210 |
| HPMC K4M Prem | 190 |
| Arginine | 100 |
| Silica | 5.5 |
| Total: | 1111 |

The formulation components were mixed in a V-blender. The resulting powder was compressed into tablets using conventional technologies.

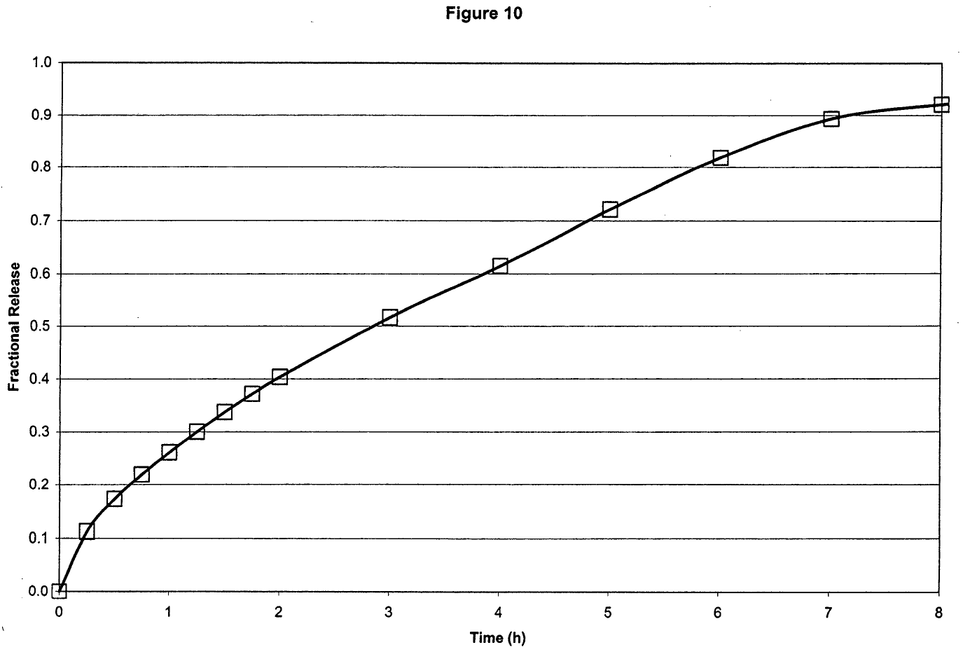

As shown in FIG. 10, the results of this Example demonstrate an in vitro release profile comprising a slight burst effect, followed by the sustained release of the remaining material. While the burst effect in this formulation produces somewhat delayed achievement of the percentage released, this formulation demonstrates in excess of 90% release over a period of 8 hours.

### Example 11

In another embodiment, the formulation comprised ibuprofen, hydroxypropyl methylcellulose (HPMC K4M), sodium carbonate, arginine, flow agents and tableting aids, in which HPMC K4M was present at a concentration of 32% by weight of ibuprofen, sodium carbonate was present at concentration of 17% by weight of the ibuprofen, and arginine was present at a concentration of 17% by weight of ibuprofen within a compressed monolithic tablet.

| Ex. 11 | mg |
|---|---|
| Ibuprofen 90 grade | 600 |
| Silica | 5.5 |
| MCC PH 105 | 210 |
| HPMC K4M Prem | 190 |
| Na₂CO₃ anhydrous | 100 |
| MCC PH 200 | 100 |
| Arginine | 100 |
| Silica | 5.5 |
| Stearic Acid | 12 |
| Total: | 1323 |

The formulation components are mixed in a V-blender. The resulting powder was compressed into tablets using conventional technologies.

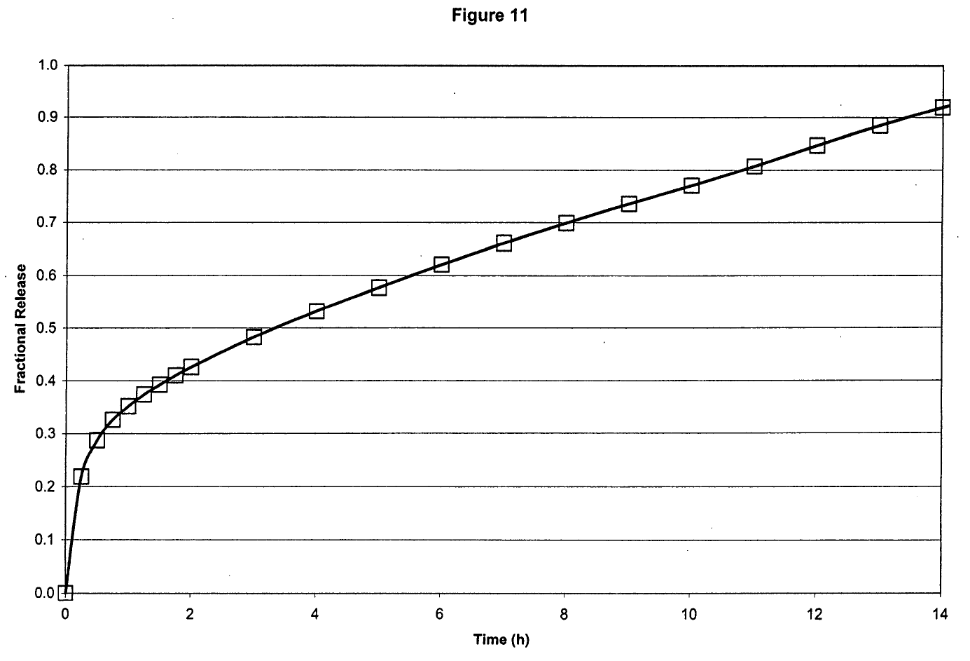

As shown in FIG. 11, the results of this Example demonstrate the in vitro release profile comprising a burst effect, followed by the sustained release of the remaining material. The initial release is greater than 20% of ibuprofen in less than two hours, and approximately 90% release over a period of 14 hours.

### Example 12

In another embodiment, the formulation comprised ibuprofen, hydroxypropyl methylcellulose (HPMC K4M), microcrystalline cellulose (MCC 105), sodium carbonate, flow agents and various tableting aids, in which HPMC K4M was present at a concentration of 32% by weight of ibuprofen, sodium carbonate was present at concentration of 17% by weight of the ibuprofen, and tableting aid, either Lactopress (12a), dicalcium phosphate (12b), or pregelatinized starch (12c), was present at a concentration of 17% by weight of ibuprofen within a monolithic tablet.

| Ex. 12a | mg | Ex. 12b | mg |
|---|---|---|---|
| Ibuprofen 90 grade | 600 | Ibuprofen 90 grade | 600 |
| Silica | 5.5 | Silica | 5.5 |
| MCC PH 105 | 210 | MCC PH 105 | 210 |
| HPMC K4M Prem | 190 | HPMC K4M Prem | 190 |
| Na₂CO₃ anhydrous | 100 | Na₂CO₃ anhydrous | 100 |
| Lactopress | 100 | Dicalcium phosphate | 100 |
| Silica | 5.5 | Silica | 5.5 |
| Stearic acid | 12 | Stearic acid | 12 |
| Total: | 1223 | Total: | 1223 |

| Ex. 12c | mg |
|---|---|
| Ibuprofen 90 grade | 600 |
| Silica | 5.5 |
| MCC PH 105 | 210 |
| HPMC K4M Prem | 190 |
| Na₂CO₃ anhydrous | 100 |
| Starch 1500 | 100 |
| Silica | 5.5 |
| Stearic acid | 12 |
| Total: | 1223 |

All ingredients were passed through a 30-mesh screen. The ibuprofen and the MCC 105 were blended in a V-blender. The resulting homogenous pre-blend was granulated with water, dried and subsequently blended with the remaining formulation components in a V-blender. The resulting powder was compressed into tablets using conventional technologies.

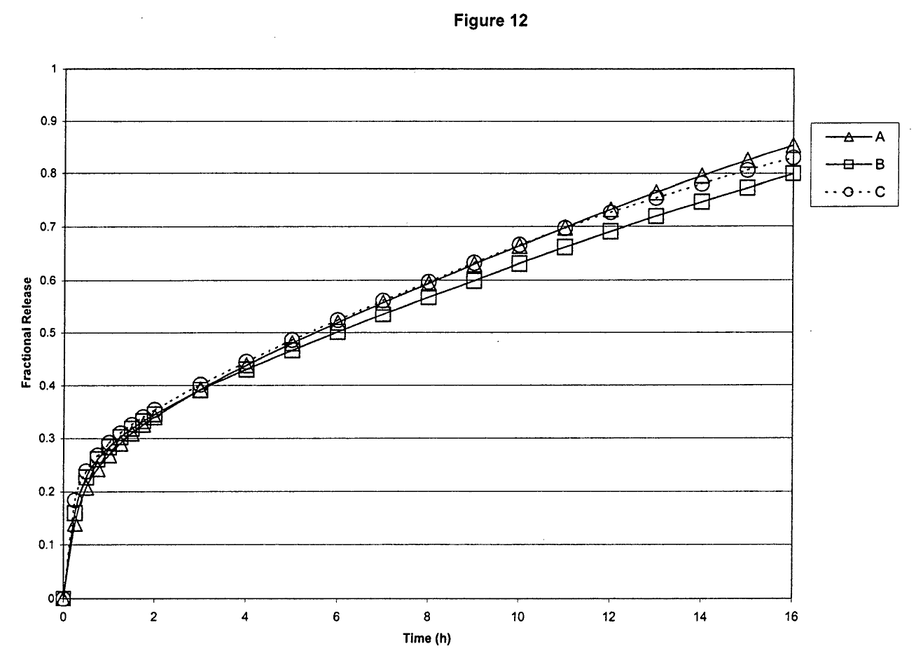

As shown in FIG. 12, the results of this Example demonstrate the invention is capable of an in vitro release profile comprising a burst effect, followed by the sustained release of the remaining material, with little or no alteration in release profile when the tableting aid selection is varied. The in vitro profile shows greater than 20% release before 2.0 hours with a constant rate release and at least 70% release by 14 hours.

### Example 13

In another embodiment, the formulation comprised ibuprofen, hydroxypropyl methylcellulose (HPMC K4M), microcrystalline cellulose (MCC 105), sodium carbonate, flow agents and various tableting aids, in which HPMC K4M was present at a concentration of 32% by weight of ibuprofen, sodium carbonate was present at concentration of 17% by weight of the ibuprofen, and croscarmellose sodium was present at a concentration of 3% by weight of ibuprofen within a monolithic tablet.

| Ex. 13 | mg |
|---|---|
| Ibuprofen 90 grade | 600 |
| Silica | 5.5 |
| MCC PH 105 | 210 |
| HPMC K4M Prem | 190 |
| Na₂CO₃ anhydrous | 100 |
| MCC PH 200 | 100 |
| Croscarmellose sodium | 18 |
| Silica | 5.5 |
| Stearic acid ˜1% | 12 |
| Total: | 1241 |

All ingredients were passed through a 30-mesh screen. The ibuprofen, silica and the MCC 105 were blended in a V-blender. The resulting homogenous pre-blend was granulated with water, dried and subsequently blended with the remaining formulation components in a V-blender. The resulting powder was compressed into tablets using conventional technologies.

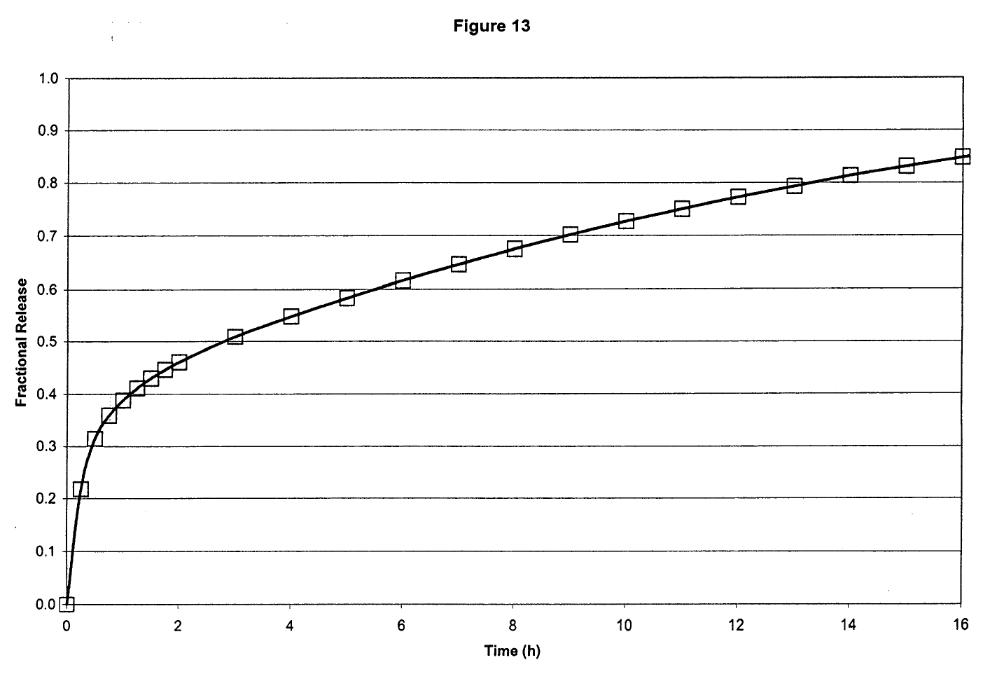

As shown in FIG. 13, the results of this Example demonstrates an in vitro release profile comprising a burst effect, followed by the sustained release of the remaining material. The in vitro profile shows greater than 20% release before 2.0 hours followed by a relatively constant rate release and at least 80% release by 14 hours.

### Example 14

In another embodiment, the formulation comprised ibuprofen, hydroxypropyl methylcellulose (HPMC K4M), microcrystalline cellulose (MCC 105), glycine, sodium carbonate, flow agents and various tableting aids, in which HPMC K4M was present at a concentration of 32% by weight of ibuprofen, sodium carbonate was present at concentration of 17% by weight of the ibuprofen, glycine was present at a concentration of 8% by weight of ibuprofen and croscarmellose sodium was present at a concentration of 6% by weight of ibuprofen within a monolithic tablet.

| Ex. 14 | mg |
|---|---|
| Ibuprofen 90 grade | 600 |
| MCC PH 105 | 200 |
| Silica | 5.5 |
| HPMC K4M Prem | 190 |
| MCC PH 200 | 100 |
| Glycine | 50 |
| Croscarmellose sodium | 35 |
| Silica | 5.5 |
| Stearic acid ˜1% | 12 |
| Total: | 1198 |

All ingredients were passed through a 30-mesh screen. The ibuprofen, silica and the MCC 105 were blended in a V-blender. The resulting homogenous pre-blend was granulated with water, dried and subsequently blended with the remaining formulation components in a V-blender. The resulting powder was compressed into tablets using conventional technologies.

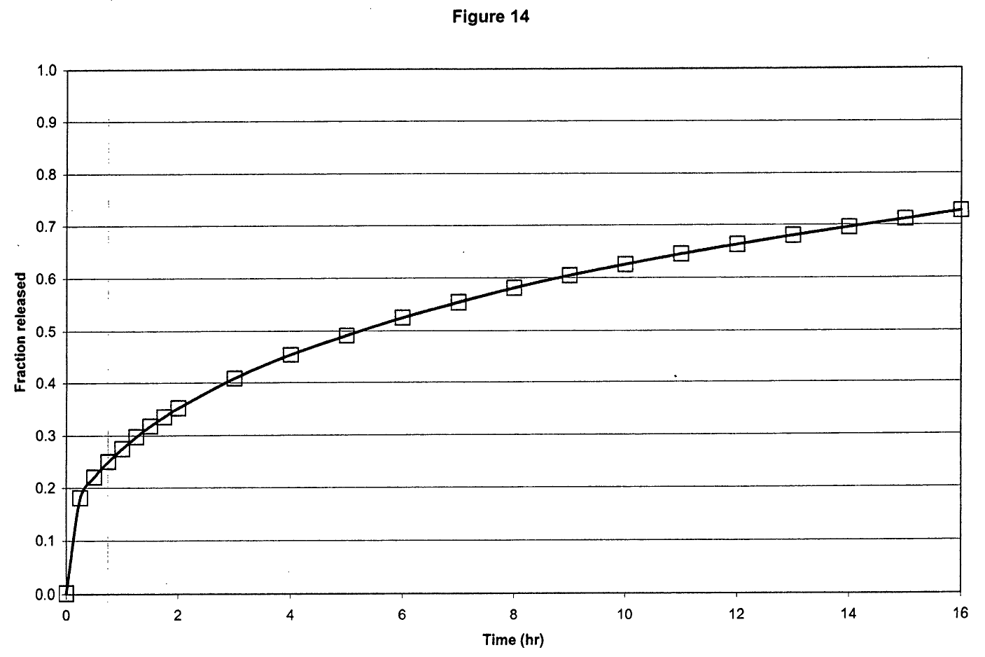

As shown in FIG. 14, the results of this Example demonstrate the invention is capable of an in vitro release profile comprising a burst effect, followed by the sustained release of the remaining material. The in vitro profile shows greater than 20% release before 2.0 hours with a constant rate release and at least 70% release by 14 hours.

### Example 15

In another embodiment, the formulation comprised ibuprofen, polyethylene oxide (PEO 301), PEO 60K, glycine, sodium carbonate, flow agents and various tableting aids, in which PEO was present at a concentration of 32% by weight of ibuprofen, sodium carbonate was present at concentration of 25% by weight of the ibuprofen, and glycine was present at a concentration of 37% by weight of ibuprofen within a monolithic tablet.

| Ex. 15 | mg |
|---|---|
| Ibuprofen | 400 |
| PEO 301 | 50 |
| PEO 60K | 75 |
| Na2CO3 | 100 |
| Glycine | 150 |
| Maltodextrin M-580 | 100 |
| Stearic acid | 10 |
| Silica | 10 |
| Total: | 895 |

All ingredients were passed through a 30-mesh screen. The ibuprofen was blended with the formulation components in a V-blender. The resulting powder was compressed into tablets using conventional technologies.

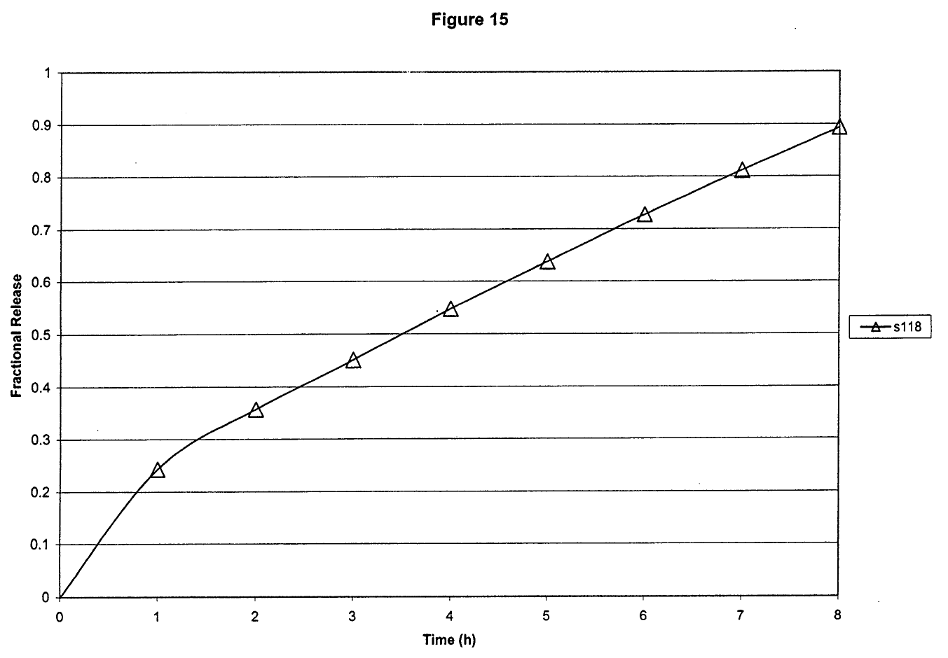

As shown in FIG. 15, the results of this Example demonstrate the invention is capable of an in vitro release profile comprising a burst effect, followed by the sustained release of the remaining material. The in vitro profile shows greater than 20% release before 2.0 hours with a constant rate release and at least 80% release by 8 hours.

### Example 16

In another embodiment, the formulation comprised ibuprofen, polyethylene oxide (PEO 301), PEO 60K, glycine, sodium carbonate, flow agents and various tableting aids, in which PEO was present at a concentration of 32% by weight of ibuprofen, sodium carbonate was present at concentration of 25% by weight of the ibuprofen, and glycine was present at a concentration of 37% by weight of ibuprofen within a monolithic tablet.

| Ex. 16 | mg |
|---|---|
| Ibuprofen | 400 |
| PEO 301 | 50 |
| PEO 60K | 50 |
| Na2CO3 | 100 |
| Glycine | 100 |
| Maltodextrin M-580 | 100 |
| Stearic acid | 10 |
| Silica | 10 |
| Total: | 820 |

All ingredients were passed through a 30-mesh screen. The ibuprofen was blended with the formulation components in a V-blender. The resulting powder was compressed into tablets using conventional technologies.

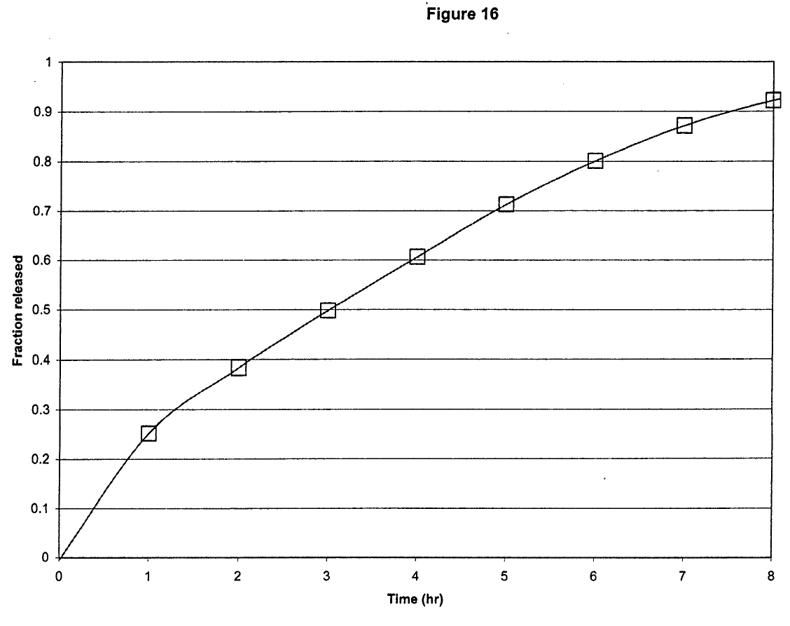

As shown in FIG. 16, the results of this Example demonstrate the invention is capable of an in vitro release profile comprising a burst effect, followed by the sustained release of the remaining material. The in vitro profile shows greater than 20% release before 2.0 hours with a constant rate release and at least 90% release by 8 hours.

### Example 17

In another embodiment, the formulation comprised ibuprofen, polyethylene oxide (PEO 301), glycine, sodium carbonate, flow agents and various tableting aids, in which PEO was present at a concentration of 25% by weight of ibuprofen, sodium carbonate was present at concentration of 25% by weight of the ibuprofen, and glycine was present at a concentration of 25% by weight of ibuprofen within a monolithic tablet.

| Ex. 17 | mg |
|---|---|
| Ibuprofen | 400 |
| PEO 301 | 100 |
| Na2CO3 | 100 |
| Glycine | 100 |
| Stearic acid | 10 |
| Total: | 710 |

All ingredients were passed through a 30-mesh screen. The ibuprofen was blended with the formulation components in a V-blender. The resulting powder was compressed into tablets using conventional technologies.

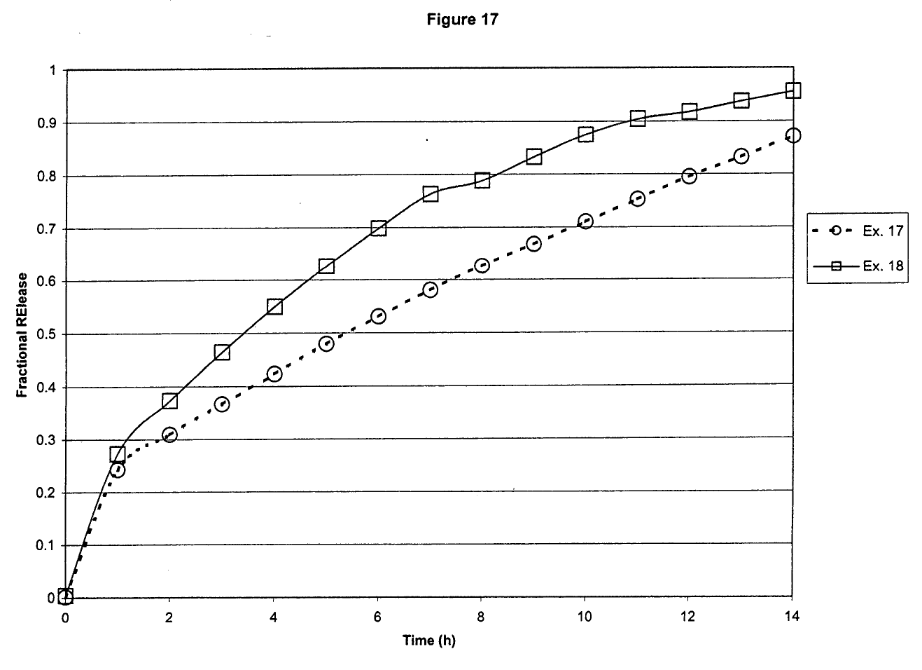

As shown in FIG. 17, the results of this Example demonstrate the invention is capable of an in vitro release profile comprising a burst effect, followed by the sustained release of the remaining material. The in vitro profile shows greater than 20% release before 2.0 hours with a constant rate release and at least 80% release by 8 hours.

### Example 18

In another embodiment, the formulation comprised ibuprofen, polyethylene oxide (PEO 301), glycine, sodium carbonate, croscarmellose sodium, flow agents and various tableting aids, in which PEO was present at a concentration of 25% by weight of ibuprofen, sodium carbonate was present at concentration of 25% by weight of the ibuprofen, and glycine was present at a concentration of 25% by weight of ibuprofen within a monolithic tablet.

| Ex. 18 | mg |
|---|---|
| Ibuprofen | 400 |
| PEO 301 | 100 |
| Na2CO3 | 100 |
| Glycine | 100 |
| Croscarmellose | 50 |
| Sodium | |
| DCP | 150 |
| Stearic acid | 10 |
| Total: | 910 |

All ingredients were passed through a 30-mesh screen. The ibuprofen was blended with the formulation components in a V-blender. The resulting powder was compressed into tablets using conventional technologies.

As shown in FIG. 17, the results of this Example demonstrate the invention is capable of an in vitro release profile comprising a burst effect, followed by the sustained release of the remaining material. The in vitro profile shows greater than 20% release before 2.0 hours with a constant rate release and at least 90% release by 8 hours.

Comparative in Vitro Data

BRUFEN RETARD is a commercially available in Europe as a sustained release formulation of ibuprofen. BRUFEN RETARD tablets are specially formulated to allow the gradual release of active substance giving stable levels and a prolonged duration of effect over the dosage interval. BRUFEN RETARD is a film coated tablet with 800 mg of ibuprofen. BRUFEN RETARD is indicated for its analgesic and anti-inflammatory effect in the treatment of rheumatoid arthritis (including juvenile rheumatoid arthritis or Still's disease), ankylosing spondylitis, and osteo-arthritis. BRUFEN RETARD is indicated in the treatment of non-articular rheumatism including fibrositis. BRUFEN RETARD is indicated in periarticular conditions such as frozen shoulder (capsulitis), bursitis, tendinitis, tenosynovitis and low-back pain. BRUFEN RETARD can also be used in soft-tissue injuries such as sprains and strains. BRUFEN RETARD is also indicated for its analgesic effect in the relief of mild to moderate pain such as dysmenorrhoea, dental, post-episiotomy pain and post-partum pain.

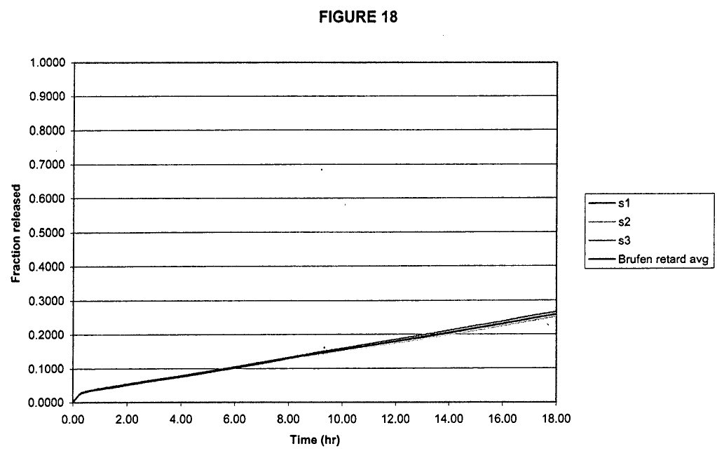

### Example 19 (FIG. 18)

BRUFEN RETARD tablet in vitro release performance was evaluated in a type II dissolution apparatus in 900 mL KH₂PO₄ buffer, pH 7.2, at 50 rpm paddle speed.

As shown in FIG. 18, the results of this Example demonstrate the in vitro data results of BRUFEN RETARD. The figure shows that BRUFEN RETARD is incapable of an in vitro release profile comprising a burst effect, followed by the sustained release of the remaining material. BRUFEN RETARD fails to deliver to release at least 20% of ibuprofen by 2.0 hours with a constant rate of release with at least 70% release at 14 hours.

### Example 20

### In Vivo Trial

In the in vivo testing, serum concentrations of subjects taking tablets comprising the modified release formulation of the present invention were compared with serum concentrations of subjects taking immediate release ibuprofen tablets (Motrin® IB 200 mg and Motrin® 600 mg). Tablets comprising the modified release formulation of the present invention demonstrated a burst effect followed by sustained release and therapeutic concentration at extended time periods that the other two immediate release formulations did not. The minimum mean serum plasma ibuprofen concentration in the blood of the subject was between 8 and 10 μg/ml for Motrin® IB.

The in vivo behavior of modified release solid dosages of 1a and 1b from Example 1 were compared to the in vivo behavior of an immediate release formulation (MOTRIN®). The open-label study involved 10 healthy male volunteers over the age of 18. Following an overnight fast of at least ten hours, each subject received either one 600 mg dose of one of the two above described modified release tablets or 200 mg every four hours for 3 doses of the immediate release formulation of MOTRIN® IB or one 600 mg tablet of MOTRIN®. 88 blood samples were taken prior to dosing and at specific intervals up to 12 hours after dosing.

The blood samples were kept in ice bath prior to centrifugation and were centrifuge as soon as possible under refrigerated condition at 35000 rpm for seven minutes. The collected plasma from each blood collection tube was aliquotted into pre-cooled labeled polypropylene tubes. The samples were kept in an ice bath, then stored frozen at minus 25° C.±10° C. until assayed.

The plasma samples were analyzed by a fully validated HPLC method. The analytes were separated by reverse phase chromatography. Evaluation of the assay was carried out by the construction of an eight point calibration curve (excluding zero concentration) covering the range of 0.400 μg/ml to 51.200 μg/ml (in human plasma) for ibuprofen. The slope and intercept of the calibration curves were determined through weighted linear regression analysis (1/conc.²). The results are depicted in FIG. 19.

TABLE 1
Summary of 90% CI

| | Reference: | | | Reference: | | |
|---|---|---|---|---|---|---|
| | D (1 × 600 mg) | | | E (3 × 200 mg) | | |
| Formulation | Cₘₐₓ | AUC₀₋ₗₐₛₜ | AUC0_{-∞} | Cₘₐₓ | AUC₀₋ₗₐₛₜ | AUC_{0-∞} |
| B (1a) | 42.4-53.8 | 96.2-115 | 97.0-116 | 67.0-85.0 | 86.9-104 | 86.3-103 |
| C (1b) | 44.7-57.0 | 96.9-116 | 98.7-119 | 70.7-90.3 | 87.5-105 | 87.7-106 |
| D | — | — | — | 140-179 | 82.3-99.2 | 80.9-97.7 |
| E | 55.9-71.5 | 101-122 | 102-124 | — | — | — |

D is a 3 × 200 mg MOTRIN ® IB
E is a 1 × 600 mg MOTRIN ®
Treatments (B & C) versus Treatment E

The systematic exposure to ibuprofen after the administration of the one 600 mg ibuprofen tablet 1a or 1b (Treatments B & C) was similar to that obtained when compared to the administration of one MOTRIN® 600 mg tablet. The peak exposure to ibuprofen from one 600 mg ibuprofen tablet 1a or 1b (Treatments A-C) was significantly lower than that from the MOTRIN® 600 mg tablet. The absorption time was modified comparing one 600 mg ibuprofen tablet 1a or 1b (Treatments B & C) with median Tₘₐₓ value of 5.0 h to a 1.5 h Tₘₐₓ of one MOTRIN® 600 mg tablet.

Treatments (B & C) versus Treatment D

The systematic exposure to ibuprofen after the administration of the one 600 mg ibuprofen tablet 1a or 1b (Treatments B & C) was similar to that obtained when compared to the administration of three MOTRIN® IB 200 mg tablets. The peak exposure to ibuprofen from one 600 mg ibuprofen tablet 1a or 1b (Treatments B & C) was significantly lower than that from three MOTRIN® IB 200 mg tablets. The absorption time was modified comparing one 600 mg ibuprofen tablet 1a or 1b (Treatments B & C) with median Tₘₐₓ value of 5.0 h to a 1.0 h Tₘₐₓ of three MOTRIN® IB 200 mg tablet.

FIG. 19 depicts the results discussed above. Treatment D shows an initial burst that falls to a valley at four hours and the second tablet is administered. This valley again happens at the eighth hour. This valley constitutes the minimum plasma concentration for ibuprofen to be considered therapeutic. A mean ibuprofen plasma concentration of about 6.4-10 μg/ml is considered the concentration of ibuprofen needed in the blood to be considered clinically effective. Treatment E shows an extreme initial burst of ibuprofen followed by a steady decline that falls below therapeutic threshold at about 6 hours.

Treatments B and C have an initial burst of ibuprofen that reaches the level of 6.4 μg/ml at about 0.5 to 1 hour and maintains the level until about hour 12. The present invention provides for a single dosage of ibuprofen that provides an initial burst similar to an immediate release formulation of ibuprofen and then provides a mean ibuprofen plasma concentration of above 6.4 μg/ml for about 12 hours.

What is claimed:

1. A solid dosage form for modified oral administration of ibuprofen comprising:
a hydrophilic polymer;
300 to 800 mg of ibuprofen in the solid dosage form uniformly dispersed in said polymer;
a dissolution additive dispersed in said hydrophilic polymer in an amount in the range of 10% to 35% by weight of the ibuprofen, said dissolution additive comprising an alkali metal salt, an amino acid having a neutral to alkaline side chain, croscarmellose or a salt thereof, or a combination of any two of such dissolution additives; and
an inert formulation additive dispersed in said hydrophilic polymer in an amount in the range of 15% to 75% by weight of the ibuprofen, said formulation additive comprising microcrystalline cellulose, silica, magnesium stearate, stearic acid, lactose, pre-gelatinized starch, dicalcium phosphate or a combination of any of them,
wherein at least 20% of the ibuprofen is released within 2 hours following oral administration or exposure to an agitated aqueous medium of a single dosage unit, then thereafter releases ibuprofen at a relatively constant rate over a period of at least 8 hours, and wherein at least 70% of the ibuprofen is released over a period of not more than 14 hours following such administration or exposure.

2. The solid dosage form of claim 1, wherein ibuprofen is present in each dosage form in an amount of about 300 mg, 400 mg or 600 mg.

3. The solid dosage form of claim 1, wherein said polymer comprises polyethylene oxide, hydroxypropyl methylcellulose or a combination thereof.

4. The solid dosage form of claim 1, wherein said polymer comprises hydroxypropyl methylcellulose with a viscosity of at least 100 cps.

5. The solid dosage form of claim 4, wherein said hydrophilic polymer comprises a first hydroxypropyl methylcellulose having a viscosity of greater than 100 cps and a second HPMC having a viscosity of about 100 cps, each at a concentration of 17% to 42% by weight of ibuprofen.

6. The solid dosage form of claim 1, wherein said dissolution additive is sodium carbonate, glycine, arginine, croscarmellose sodium or a combination thereof.

7. The solid dosage form of claim 1, where said inert formulation additive comprises microcrystalline cellulose present at a concentration at 17% to about 33% by weight of the ibuprofen.

8. The solid dosage form of claim 7, wherein said inert formulation additive comprises a first microcrystalline cellulose having particle size of about 20 μm and a second MCC having a particles size of about 180 μm, each of which is present at a concentration at 17% to about 33% by weight of the ibuprofen.

9. The solid dosage form of claim 1, wherein said solid dosage form demonstrates a mean serum ibuprofen concentration in a subject greater than or equal to 6.4 μg/ml within two hours of administration, and wherein said solid dosage form also demonstrates a mean serum ibuprofen concentration in a subject greater than or equal to 6.4 μg/ml for at least 8 hours after administration.

10. A modified release tablet, comprising:
ibuprofen in an amount in the range of 300 mg to 800 mg per tablet;
a hydrophilic polymer;
a dissolution additive at a concentration of from 10% to 35% by weight of the ibuprofen comprising alkali metal salts, an amino acid possessing neutral-to-alkaline side chain, croscarmellose or a salt thereof or a combination thereof; and
an inert formulation additive comprising microcrystalline cellulose, silicified microcrystalline cellulose, dicalcium phosphate, lactose, pre-gelatinized starch or mixtures thereof, said inert formulation additive being present in said dosage in an amount of 15% to about 75% by weight of the ibuprofen,
wherein said tablet demonstrates a mean serum ibuprofen concentration in a subject greater than or equal to 6.4 μg/ml within two hours of administration, and wherein said tablet also demonstrates a mean serum ibuprofen concentration in a subject greater than or equal to 6.4 μg/ml for at least 8 hours after administration.

11. The tablet of claim 10,
wherein the hydrophilic polymer comprises hydroxypropyl methylcellulose at a concentration of 17% to 42% by weight of ibuprofen;
wherein ibuprofen is present in an amount of about 600 mg and dispersed uniformly in said polymer;
wherein said dissolution additive is sodium carbonate uniformly dispersed in said polymer; and
wherein said formulation additive is two differing particle sizes of microcrystalline cellulose dispersed in said polymer, each at 15% to 50% by weight of the ibuprofen.

12. The tablet of claim 10,
wherein the hydrophilic polymer comprises hydroxypropyl methylcellulose at a concentration of 17% to 42% by weight of ibuprofen;
wherein ibuprofen is present in an amount of about 600 mg and is dispersed uniformly in said polymer;
wherein said dissolution additive is glycine uniformly dispersed in said polymer at a concentration of 10% to 15% by weight of the ibuprofen.

13. The tablet of claim 10,
wherein the hydrophilic polymer comprises hydroxypropyl methylcellulose at a concentration of 17% to 42% by weight of ibuprofen;
wherein ibuprofen is present in an amount of about 600 mg and is dispersed uniformly in said polymer;
wherein said dissolution additive is glycine uniformly dispersed in said polymer at a concentration of 10% to 15% by weight of the ibuprofen;
wherein said formulation additive is two differing particle sizes of microcrystalline cellulose dispersed in said polymer, each at 15% to 50% by weight of the ibuprofen.

14. The tablet of claim 10,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein the dissolution additive is glycine uniformly dispersed in said polymer at a concentration of 5% to 35% by weight of the ibuprofen;
wherein the formulation additive is two differing particle sizes of microcrystalline cellulose dispersed in said polymer, each at 15% to 50% by weight of the ibuprofen.

15. The tablet of claim 10,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 300 mg to 800 mg ibuprofen dispersed uniformly in said polymer;
wherein the dissolution additive is glycine uniformly dispersed in said polymer at a concentration of 5% to 35% by weight of the ibuprofen and croscarmellose sodium uniformly dispersed in said polymer at a concentration of 1% to 15% by weight of the ibuprofen.

16. The tablet of claim 10,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 300 mg to 800 mg ibuprofen dispersed uniformly in said polymer;
wherein the dissolution additive is glycine uniformly dispersed in said polymer at a concentration of 5% to 35% by weight of the ibuprofen and croscarmellose sodium uniformly dispersed in said polymer at a concentration of 1% to 15% by weight of the ibuprofen;
wherein the formulation additive is two differing particle sizes of microcrystalline cellulose dispersed in said polymer, each at 15% to 50% by weight of the ibuprofen.

17. The tablet of claim 10,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 300 mg to 800 mg ibuprofen dispersed uniformly in said polymer;
wherein the dissolution additive is sodium carbonate uniformly dispersed in said polymer at a concentration of 5% to 35% by weight of the ibuprofen;
wherein the formulation additive is two differing particle sizes of microcrystalline cellulose dispersed in said polymer, each at 15% to 50% by weight of the ibuprofen.

18. The tablet of claim 10,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 300 mg to 800 mg ibuprofen dispersed uniformly in said polymer;
wherein the dissolution additive is sodium carbonate uniformly dispersed in said polymer at a concentration of 5% to 35% by weight of the ibuprofen, and croscarmellose sodium uniformly dispersed in said polymer at a concentration of 1% to 15% by weight of the ibuprofen;
wherein the formulation additive is two differing particle sizes of microcrystalline cellulose dispersed in said polymer, each at 15% to 50% by weight of the ibuprofen.

19. The tablet of claim 10,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 420% by weight of ibuprofen;
wherein 300 mg to 800 mg ibuprofen dispersed uniformly in said polymer;
wherein the dissolution additives are sodium carbonate uniformly dispersed in said polymer at a concentration of 5% to 35% by weight of the ibuprofen, glycine uniformly dispersed in said polymer at a concentration of 5% to 35% by weight of the ibuprofen, and croscarmellose sodium uniformly dispersed in said polymer at a concentration of 1% to 15% by weight of the ibuprofen;
wherein the formulation additive is two differing particle sizes of microcrystalline cellulose dispersed in said polymer, each at 15% to 50% by weight of the ibuprofen.

20. A method of maintaining a mean plasma ibuprofen concentration of at least 6.4 μg/ml over a time period of 2 to 8 hours in a patient, comprising:
administering a single dosage of the solid dosage form according to claim 1.

21. The method for providing immediate and extended release of ibuprofen to a subject, comprising:
administering to a subject in a single dose of a modified release tablet comprising,
ibuprofen in an amount in the range of 300 mg to 800 mg per tablet;
a hydrophilic polymer;
a dissolution additive at a concentration of from 10% to 35% by weight of the ibuprofen comprising alkali metal salts, an amino acid possessing neutral-to-alkaline side chain, croscarmellose or a salt thereof or a combination thereof; and
an inert formulation additive comprising microcrystalline cellulose, silicified microcrystalline cellulose, dicalcium phosphate, lactose, pre-gelatinized starch or mixtures thereof, said inert formulation additive being present in said dosage in an amount of 15% to about 75% by weight of the ibuprofen,
wherein said tablet demonstrates a mean serum ibuprofen concentration in a subject greater than or equal to 6.4 μg/ml within two hours of administration, and wherein said tablet also demonstrates a mean serum ibuprofen concentration in a subject greater than or equal to 6.4 μg/ml for at least 8 hours after administration.

22. The method according to claim 21,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 300 mg to 800 mg ibuprofen dispersed uniformly in said polymer;
wherein the dissolution additives are sodium carbonate uniformly dispersed in said polymer at a concentration of 5% to 35% by weight of the ibuprofen, glycine uniformly dispersed in said polymer at a concentration of 50% to 35% by weight of the ibuprofen, and croscarmellose sodium uniformly dispersed in said polymer at a concentration of 1% to 15% by weight of the ibuprofen;
wherein the formulation additive is two differing particle sizes of microcrystalline cellulose dispersed in said polymer, each at 15% to 50% by weight of the ibuprofen.

23. The method according to claim 21,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 300 mg to 800 mg ibuprofen dispersed uniformly in said polymer;
wherein the dissolution additive is sodium carbonate uniformly dispersed in said polymer at a concentration of 5% to 35% by weight of the ibuprofen, and croscarmellose sodium uniformly dispersed in said polymer at a concentration of 1% to 15% by weight of the ibuprofen;
wherein the formulation additive is two differing particle sizes of microcrystalline cellulose dispersed in said polymer, each at 15% to 50% by weight of the ibuprofen.

24. The method according claim 21,
wherein said hydrophilic polymer comprises hydroxypropyl methylcellulose having two differing viscosities, selected from the group consisting of HPMC 100 cps, HPMC 4000 cps, HPMC 15000 cps, and HPMC 100000 cps, each at a concentration of 17% to 42% by weight of ibuprofen;
wherein 600 mg ibuprofen is dispersed uniformly in said polymer; and
wherein the dissolution additive is sodium carbonate uniformly dispersed in said polymer at a concentration of 10% to 35% by weight of the ibuprofen, and glycine uniformly dispersed in said polymer at a concentration of 1% to 15% by weight of the ibuprofen.
